(19) United States
(12) Patent Application Publication
(10) Pub. No.: US 2025/0127695 A1
(43) Pub. Date: Apr. 24, 2025

(54) Use of Branched Medium-Chain Glycerol Ethers as Active Antidandruff Ingredients

(71) Applicant: Henkel AG & Co. KGaA, Duesseldorf (DE)

(72) Inventors: Petra Westphal, Neu Wulmstorf (DE); Rainer Simmering, Grevenbroich (DE); Sabine Gruedl, Erkelenz (DE); Thomas Schroeder, Hamburg (DE); Dirk Hentrich, Hamburg (DE)

(21) Appl. No.: 18/906,654

(22) Filed: Oct. 4, 2024

Related U.S. Application Data
(63) Continuation of application No. PCT/EP2023/050374, filed on Jan. 10, 2023.

(30) Foreign Application Priority Data
Apr. 7, 2022 (DE) 102022203486.4

(51) Int. Cl. A61K 8/34; A61Q 5/00; A61Q 5/02

(52) U.S. Cl. CPC A61K 8/345; A61Q 5/006; A61Q 5/02

(57) ABSTRACT

Compounds of the following formula (I) R¹—O—[CH₂—CH(OH)—CH₂—O]n-H (I), in which R¹ is a branched C₆-C₁₆ alkyl, alkenyl or alkynyl group and n is a number of 1-3, are suitable for use as active antidandruff ingredient and as active skin-soothing ingredient for nontherapeutic cosmetic use in cosmetic formulations.

### FIELD OF THE INVENTION

The present invention relates to the cosmetic use of specific glycerol ethers as active anti-dandruff ingredients and as active skin-soothing ingredients, and in particular in the cosmetic treatment of dandruff conditions associated with the bacterial colonization and proliferation of yeast fungi of the genus Malassezia on hair-covered skin surfaces.

Even mild dandruff on the scalp or other hair-covered areas of the body is perceived as a sign of a lack of care and hygiene. The formation of dandruff is usually accompanied by painful itching, which can cause scratching reactions and lead to damage to the affected areas of skin. Skin damage in turn often forms the basis for infections and pathogens.

Dandruff formation can be promoted by various factors such as hereditary predisposition, a tendency for increased sebum production (seborrhea), hormone fluctuations, stress, climatic conditions, or improper hair care. However, the decisive contribution to dandruff formation is made by the colonization of the scalp with yeast fungi of the genus Malassezia. In the fight against dandruff, efforts have therefore always been made to reduce and/or eliminate the bacterial colonization of the scalp with Malassezia.

### BACKGROUND OF THE INVENTION

For this purpose, numerous cosmetically tolerated and effective active anti-dandruff ingredients are known from the prior art, such as piroctone olamine, climbazole, zinc pyrithione or other zinc salts, sulfur, and/or selenium sulfide.

The effect of these active anti-dandruff ingredients is mainly based upon their fungicidal properties.

However, in view of the growing importance of using raw materials and energy more sustainably, efforts are increasingly being made to use only those cosmetic raw materials for cosmetic products that can be obtained from renewable raw materials using as little energy as possible. However, a reduction in quantity or even a complete replacement of undesirable or problematic substances can only be made if alternatives are available that also have the desired properties for the intended use. This also applies to active anti-dandruff ingredients.

Various plant extracts based upon natural materials are known as active anti-dandruff ingredients—for example, arnica, birch, burdock root, beard lichen, poplar, nettle, myrtle, or hazel root extracts. However, with respect to their immediate and/or long-lasting effectiveness, the extracts cannot completely replace the aforementioned conventional active anti-dandruff ingredients.

There is therefore still a need for compounds with good efficacy against dandruff which are obtainable from natural sources.

It has been found that specific biobased glycerol ethers are ideally suited for this purpose.

WO 2021/237207 A1 describes the preparation of biobased glycerol ethers which can be prepared from sustainable, renewable, plant-based raw materials and which—just like other branched glycerol ethers, e.g., ethylhexyl glycerol—are excellently suited as preservatives in cosmetic or pharmaceutical compositions or as odor control agents in deodorants.

In addition, according to EP 3944852, some glycerol ethers, such as, for example, ethylhexyl glycerol, are suitable as boosters for enhancing the anti-dandruff efficacy of piroctone olamine in cosmetic compositions.

However, it is not known to use medium-chain glycerol ethers as an active anti-dandruff ingredient, and in particular as the sole active anti-dandruff ingredient, in cosmetic compositions for the treatment of dandruff conditions.

### BRIEF SUMMARY OF THE INVENTION

The object of the present invention was to find biodegradable active ingredients which are easily accessible from natural sources and are well tolerated by the skin, which can be incorporated into a plurality of cosmetic agents without difficulty and with long-term stability, and which reduce, prevent, and/or eliminate excessive colonization of hair-covered body surfaces, and in particular the scalp, by fungi of the genus Malassezia.

It was surprisingly found that the cosmetic use of very specific medium-chain glycerol ethers enables effective treatment of dandruff conditions, and in particular those associated with colonization and/or proliferation of yeast fungi of the genus Malassezia.

The use according to the invention of at least one specific medium-chain glycerol ether as the sole active anti-dandruff ingredient in cosmetic compositions resulted in a significant reduction in the quantity of yeasts of the genus Malazessia, and consequently in a reduction in dandruff.

Furthermore, the use of at least one specific medium-chain glycerol ether according to the invention was found to provide additional advantages in maintaining the endogenous barrier—in particular, on sensitive scalps (where there is a disturbed barrier and increased sebum production). The use according to the invention of at least one specific medium-chain glycerol ether has a balancing and soothing effect on sensitive scalps.

### BRIEF DESCRIPTION OF THE DRAWINGS

FIG. 1 shows the results of quantitative suspension test of Malassezia furfur over application times of 10 and 60 minutes using various concentrations methylheptyl glyceryn (MHG) and a water control.

FIG. 2 shows the gene expression of transglutaminase1 (TGM1), filaggrin (FLG), decorin (DCN), involucrin (IVL), and loricrin (LOP) after treatment with various concentrations of MHG.

FIG. 3 shows the IL 8 protein release percentage following the application of various concentrations of MHG as well as a control.

### DETAILED DESCRIPTION OF THE INVENTION

The present invention is therefore characterized by the following statements:

1. A non-therapeutic use of at least one compound of the following formula (I)

R¹—O—[CH₂—CH(OH)—CH₂—O]ₙ—H (I),

wherein
R¹ is a branched C₆-C₁₆ alkyl, alkenyl, or alkynyl group, and
n is a number from 1 to 3, as an active anti-dandruff ingredient.
   2. A non-therapeutic use of at least one compound of the following formula (I)

R¹—O—[CH₂—CH(OH)—CH₂—O]ₙ—H (I),

wherein
R¹ is a branched C₆-C₁₆ alkyl, alkenyl, or alkynyl group, and
n is a number from 1 to 3,
as an active skin-soothing ingredient.
   3. The use according to one of statements 1 or 2, wherein n is the number 1.
   4. The use according to one of the preceding statements, wherein R¹ is a branched C₆-C₁₂ alkyl, alkenyl, or alkynyl group.
   5. The use according to one of the preceding statements, wherein R¹ is a branched C₆-C₁₀ alkyl, alkenyl, or alkynyl group.
   6. The use according to one of the preceding statements, wherein R¹ is a branched C₈ alkyl, alkenyl, or alkynyl group.
   7. The use according to one of the preceding statements, wherein the compound according to formula (I) is H₃C—(CH₂)₅—CH(CH₃)—O—CH₂—CH(OH)—CH₂OH (methylheptyl glycerin).
   8. The use according to one of the preceding statements for reducing or preventing the bacterial colonization of hair-covered skin surfaces with yeast fungi of the genus Malassezia.
   9. The use according to statement 8 for reducing or preventing the bacterial colonization of hair-covered skin with yeast fungi of the genus Malassezia furfur and/or Malassezia globosa.
   10. The use according to one of the preceding statements in cosmetic formulations.
   11. The use according to one of the preceding statements in cosmetic formulations for treating the hair or the scalp.
   12. The use according to one of statements 10 or 11, wherein the cosmetic formulation is a shampoo.
   13. The use according to statement 12, wherein the cosmetic formulation contains, relative to its total weight, 0.05-20 wt % of at least one surfactant.
   14. The use according to one of statements 10 or 11, wherein the cosmetic formulation is a hair care product.
   15. The use according to one of statements 10 through 14, wherein the cosmetic formulation contains 0.05-5 wt % of a compound according to formula (I), relative to the total weight of the cosmetic formulation.
   16. The use according to statement 15, wherein the cosmetic formulation contains a compound according to formula (I) as the sole active anti-dandruff ingredient.

Compounds according to formula (I) are preferably used in cosmetic formulations, and in particular in cosmetic formulations for treating the hair or the scalp.

Cosmetic formulations suitable according to the invention can be present in any galenic form customary for topical application—for example, in the form of aqueous or hydroalcoholic solutions, oil-in-water emulsions, water-in-oil emulsions, triple emulsions (O/W/O or W/O/W), aqueous gels, dispersions of fatty phases, or solid ingredients in aqueous phases.

These compositions can be prepared by the customary methods.

In addition, cosmetic formulations suitable according to the invention can be more or less liquid and have the appearance of a white or colored cream, an ointment, a milk, a lotion, a serum, a paste, a foam, a shampoo, a gel, or a stick.

Cosmetic formulations suitable according to the invention contain at least one compound according to formula (I) preferably in a physiologically tolerated medium, preferably in water, and optionally in a physiologically tolerated organic solvent, which may be selected, for example, from C2-C8 alcohols, and in particular C2-C6 alcohols, such as ethanol, isopropanol, propanol, butanol, propylene glycol, isoprene glycol, butylene glycol, glycerol, sorbitol, or mixtures thereof.

Particularly preferably, cosmetic formulations suitable according to the invention comprise, as a physiologically tolerated medium, 30-98 wt %, more preferably 40-95 wt %, and in particular 50-90 wt % water, relative to the total weight of the cosmetic formulation.

If present, the quantity of the aforementioned organic solvents in the cosmetic formulations suitable according to the invention (relative to their total weight) is preferably 0-60 wt %, more preferably 0.05-55 wt %, and particularly preferably 0.1-50 wt %.

Cosmetic formulations suitable according to the invention, in addition to at least one compound according to formula (I) and a physiologically tolerated medium, can contain further auxiliaries and active ingredients customary in the cosmetic sector, e.g., oils, waxes, butters, fats, pigments, fillers, dyes, surfactants, emulsifiers, UV protectants, polymers, thickeners, film formers, preservatives, fragrances, bactericides, odor absorbers, antioxidants, pH adjusters, sequestering agents, wherein they are preferably free of other compounds known for their anti-dandruff effect.

Preferred cosmetic formulations according to the invention contain, in a physiologically tolerated medium, in addition to at least one compound according to formula (I), at least one further cosmetic ingredient which is preferably selected from vegetable or mineral oils, fatty alcohols, fatty acid esters, waxes, cationic, amphoteric, zwitterionic, non-ionic, and/or anionic surfactants, cationic, amphoteric, zwitterionic, non-ionic, and/or anionic polymers, sunscreens, antioxidants, anti-hair loss agents, plant extracts, proteins, protein hydrolysates, pearlescent agents, opacifiers, emollients, hydroxy acids, fillers, silicones, fragrances, alkalizing agents, acidifying agents, preservatives, sequestering agents, colorants, vitamins, amino acids, or any mixtures thereof, wherein they are preferably free of other compounds known for their anti-dandruff effect.

These further cosmetic ingredients can be used in the cosmetic formulations suitable according to the invention in customary quantities and amount, for example, to 0.01-30 wt % of the total weight of the respective cosmetic formulation.

According to a preferred embodiment, at least one compound according to formula (I) is used as the sole active anti-dandruff ingredient in the respective cosmetic formulation.

According to a further preferred embodiment, the pH value of cosmetic formulations suitable according to the invention is 2-9, preferably 2.5-8.5, and in particular 3-8.

According to a further preferred embodiment, the cosmetic formulation is a composition for treating hair, which may be in the form of a shampoo, a cream, an aerosol foam, a paste, a gel, an emulsion, a lotion, a tonic, a stick, or a bar of soap.

In a very particularly preferred embodiment, the cosmetic formulation is a shampoo and preferably contains, relative to its total weight, 0.05-20 wt % of at least one surfactant.

Within this embodiment, shampoos are preferred which contain at least one anionic surfactant or a mixture of anionic and amphoteric and/or zwitterionic surfactants.

Very particularly preferred are shampoos which contain, relative to their total weight,
1-15 wt %, more preferably 2-13 wt %, and particularly preferably 3-12 wt % of at least one anionic surfactant and
0-10 wt %, more preferably 0.5-8.5 wt %, and particularly preferably 1-7 wt % of at least one amphoteric and/or zwitterionic surfactant.

In addition, shampoos within this embodiment may contain non-ionic surfactants in a proportion by weight of 0-10 wt %, more preferably 0.5-8.5 wt %, and particularly preferably 1-7 wt % of the total weight of the shampoo.

All anionic surface-active substances suitable for use on the human body are suitable as anionic surfactants. These are characterized by a water-solubilizing, anionic group such as a carboxylate, sulfate, sulfonate, or phosphate group and a lipophilic alkyl group having approximately 8 to 30 C atoms. In addition, glycol ether or polyglycol ether groups, ester, ether and amide groups, and hydroxyl groups can be contained in the molecule. Examples of suitable anionic surfactants are, in each case in the form of the sodium, potassium, and ammonium and mono-, di-, and trialkanol ammonium salts having 2 to 4 carbon atoms in the alkanol group,
linear and branched fatty acids having 8 to 30 C atoms (soaps);
ether carboxylic acids of the formula R—O—(CH₂—CH₂O)ₓ—CH₂—COOH, in which R is a linear alkyl group having 8 to 30 C atoms, and x=0 or is 1 to 16;
acyl sarcosides having 8 to 24 C atoms in the acyl group;
acyl taurides having 8 to 24 C atoms in the acyl group;
acyl isethionates having 8 to 24 C atoms in the acyl group;
sulfosuccinic acid mono and dialkyl esters having 8 to 24 C atoms in the alkyl group and sulfosuccinic acid monoalkylpolyoxyethyl esters having 8 to 24 C atoms in the alkyl group and 1 to 6 oxyethyl groups;
linear alkane sulfonates having 8 to 24 C atoms;
linear α-olefin sulfonates having 8 to 24 C atoms;
alpha-sulfo fatty acid methyl esters of fatty acids having 8 to 30 C atoms;
alkyl sulfates and alkyl polyglycol ether sulfates of the formula R—O(CH2-CH2O)x-SO3H, in which R is a preferably linear alkyl group having 8 to 30 C atoms, and x=0 or 1 to 12;
mixtures of surface-active hydroxysulfonates according to DE-A-37 25 030;
sulfated hydroxyalkyl polyethylene and/or hydroxyalkylene propylene glycol ethers according to DE-A-37 23 354;
sulfonates of unsaturated fatty acids having 8 to 24 C atoms and 1 to 6 double bonds according to DE-A-39 26 344;
esters of tartaric acid and citric acid with alcohols which represent addition products of about 2-15 molecules ethylene oxide and/or propylene oxide to fatty alcohols having 8 to 22 C atoms;
alkyl and/or alkenyl ether phosphates of the formula

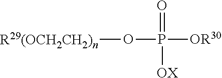

in which R29 preferably represents an aliphatic hydrocarbon functional group having 8 to 30 carbon atoms, R30 represents hydrogen, a functional group (CH₂CH₂O)ₙR29, or X, n represents numbers from 1 to 10, and X represents hydrogen, an alkali metal, or alkaline-earth metal, or NR31 R32R33R34, with R31 to R34 representing, independently of each other, a C1 to C4 hydrocarbon functional group;
sulfated fatty acid alkylene glycol esters of the formula

R35CO(AlkO)ₙSO₃M

in which R35CO- represents a linear or branched, aliphatic, saturated and/or unsaturated acyl functional group having 6 to 22 C atoms, Alk represents CH2CH2, CHCH3CH2,
and/or CH2CHCH3, n represents numbers from 0.5 to 5, and M represents a cation, as described in DE-OS 197 36 906.5;
monoglyceride sulfates and monoglyceride ether sulfates of the formula (III) as described, for example, in EP-B1 0 561 825, EP-B1 0 561 999, DE-A1 42 04 700, or A. K. Biswas et al. in J. Am. Oil. Chem. Soc. 37, 171 (1960) and F. U. Ahmed in J. Am. Oil. Chem. Soc. 67, 8 (1990):

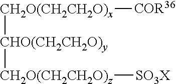

in which R36CO represents a linear or branched acyl functional group having 6 to 22 carbon atoms, x, y, and z represent 0 or numbers from 1 to 30, and preferably 2 to 10, in total, and X represents an alkali metal or alkaline-earth metal. Typical examples of monoglyceride (ether) sulfates suitable in the sense of the invention are the reaction products of lauric acid monoglyceride, coconut fatty acid monoglyceride, palmitic acid monoglyceride, stearic acid monoglyceride, oleic acid monoglyceride, and tallow fatty acid monoglyceride and also the ethylene oxide adducts thereof with sulfur trioxide or chlorosulfonic acid in the form of the sodium salts thereof. Monoglyceride sulfates of formula (III), in which R36CO represents a linear acyl functional group having 8 to 18 carbon atoms, are preferably used.

Preferred anionic surfactants are alkyl sulfates, alkyl polyglycol ether sulfates, and ether carboxylic acids having 10 to 18 C atoms in the alkyl group and up to 12 glycol ether groups in the molecule, sulfosuccinic acid mono- and dialkyl esters having 8 to 18 C atoms in the alkyl group, and sulfosuccinic acid monoalkyl polyoxyethyl esters having 8 to 18 C atoms in the alkyl group and 1 to 6 oxyethyl groups.

Particularly preferred anionic surfactants are straight chain or branched alkyl ether sulfates which contain an alkyl functional group having 8 to 18 and in particular 10 to 16 C atoms and 1 to 6 and in particular 2 to 4 ethylene oxide units—preferably sodium lauryl ether sulfate having 2 ethylene oxide units.

Further particularly preferred anionic surfactants are straight chain or branched alkyl sulfates which contain an alkyl radical having 8 to 18 and in particular 10 to 16 C atoms. The sodium, magnesium, and/or triethanolamine salts of linear or branched lauryl, tridecyl, and/or myristyl sulfates are particularly preferred.

Suitable amphoteric or zwitterionic surfactants carry both a cationic and an anionic charge in the molecule. Preferably, amphoteric surfactants have at least one quaternary ammonium group and at least one —COO⁻ or —SO₃⁻ group in the molecule, in addition to a preferably C8-C24 alkyl or acyl group. Furthermore, they are capable of forming internal salts. Particularly preferred amphoteric surfactants are betaines such as the N-alkyl-N,N-dimethyl ammonium glycinates, e.g., cocoalkyl-dimethyl ammonium glycinate, N-acyl-aminopropyl-N,N-dimethyl ammonium glycinate, e.g., cocoacylaminopropyl-dimethyl ammonium glycinate, and 2-alkyl-3-carboxymethyl-3-hydroxyethyl-imidazolines having in each case 8 to 18 carbon atoms in the alkyl or acyl group, and cocoacylaminoethyl hydroxyethyl carboxymethyl glycinate. A preferred amphoteric surfactant is the fatty acid amide derivative known under the INCI name, Cocamidopropyl Betaine.

Suitable non-ionic surfactants/emulsifiers preferably include:
addition products of 4 to 30 mol of ethylene oxide and/or 0 to 5 mol of propylene oxide to linear fatty alcohols having 8 to 22 C atoms, to fatty acids having 12 to 22 C atoms, and to alkylphenols having 8 to 15 C atoms in the alkyl group,
ethylene oxide and polyglycerol addition products to methyl glucoside fatty acid esters, fatty acid alkanolamides, and fatty acid glucamides,
amine oxides,
sorbitan fatty acid esters and addition products of ethylene oxide to sorbitan fatty acid esters, such as polysorbates,
fatty acid alkanolamides of the following general formula

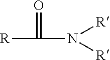

in which R preferably signifies a linear or branched, saturated or unsaturated alkyl or alkenyl functional group having 8 to 24 carbon atoms, and the functional groups R′ denote hydrogen or the group —(CH₂)ₙOH, in which n signifies the number 2 or 3, with the proviso that at least one of the functional groups R′ denotes the aforementioned functional group —(CH₂)ₙOH,
sugar fatty acid esters and addition products of ethylene oxide to sugar fatty acid esters,
addition products of ethylene oxide to fatty acid alkanolamides and fatty amines,
alkyl(oligo)glucosides,
mixtures of alkyl(oligo)glucosides and fatty alcohols—for example, the commercially available product, Montanov®68,
addition products of 5 to 60 mol ethylene oxide to castor oil and hydrogenated castor oil,
partial esters of polyols having 3-6 carbon atoms with saturated fatty acids having 8 to 22 C atoms,
sterols. Sterols are understood to refer to a group of steroids that bear a hydroxy group at the C atom 3 of the steroid structure, and are isolated both from animal tissue (zoosterols) and from vegetable fats (phytosterols). Examples of zoosterols include cholesterol and lanosterol. Examples of suitable phytosterols include ergosterol, stigmasterol, and sitosterol. Sterols known as mycosterols are also isolated from fungi and yeasts.
phospholipids. These are understood to mean principally the glucose phospholipids, which are obtained, for example, as lecithins or phosphatidylcholines from, for example, egg yolk or plant seeds (e.g., soybeans).

Alkyl(oligo)glycosides can be selected from compounds of the general formula RO-[G]ₓ, in which [G] is preferably derived from aldoses and/or ketoses having 5-6 carbon atoms, and preferably from glucose.

The index number x denotes the degree of oligomerization (DP), i.e., the distribution of mono- and oligoglycosides. The index number x preferably has a value in the range of 1 to 10, and particularly preferably in the range of 1 to 3, wherein it need not be a whole number, but can be a fractional number which can be determined by analysis. Particularly preferred alkyl(oligo)glycosides have a degree of oligomerization between 1.2 and 1.5. The functional group R preferably denotes at least one alkyl and/or alkenyl functional group having 4 to 24 C atoms.

Especially preferred alkyl(oligo)glycosides are compounds that are known under the INCI designations, Caprylyl/Capryl Glucoside, Decyl Glucoside, Lauryl Glucoside, and Coco Glucoside, or mixtures thereof.

Amine oxides may be selected from at least one compound of the general formulae (I) or (II)

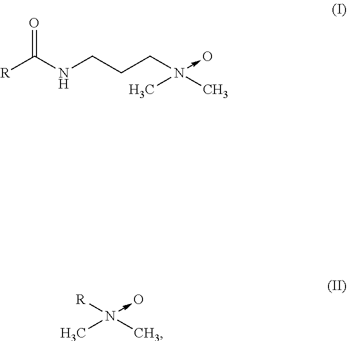

in which R in each case represents a straight-chain or branched, saturated or monounsaturated or polyunsaturated alkyl or alkenyl group having 6 to 24 carbon atoms, and preferably having 8 to 18 carbon atoms.

Particularly preferred are the surfactants of the aforementioned formulae (I) or (II) that are known under the INCI designations, Cocamine Oxide, Lauramine Oxide, and/or Cocamidopropylamine Oxide, and are commercially available from a number of suppliers.

Particularly preferred are alkyl(oligo)glucosides, and in particular compounds known by the INCI designations, Caprylyl/Capryl Glucoside, Decyl Glucoside, Lauryl Glucoside, and/or Coco Glucoside.

In a further very particularly preferred embodiment, the cosmetic formulation is a hair care product.

Hair care products are understood to mean both customary rinse-off and leave-on compositions. Preferably, compounds according to formula (I) can be used in hair rinses, serums, or tonics.

Compounds according to formula (I) are preferably used in cosmetic formulations in an amount of 0.05-5 wt %, relative to the total weight of the respective cosmetic formulation. Preferably, compounds according to formula (I) of 0.1-4 wt %, more preferably 0.15-3 wt %, further preferably 0.2-2.5 wt %, and in particular 0.3-2 wt % are used.

Cosmetic formulations comprising at least one compound according to formula (I) are preferably applied to the scalp and hair at room temperature (25° C.) and left to act for a period of 20 seconds to 30 minutes—preferably at ambient temperature.

During or after application of the cosmetic formulation, the scalp can be massaged with the hands or with a suitable aid in order to achieve optimum distribution of the cosmetic formulation and/or better action of the cosmetic formulation on the scalp.

After the application time, it can be advantageous if the cosmetic formulation is preferably rinsed off with water, and the hair is then dried.

According to a preferred embodiment, the cosmetic formulation containing at least one compound according to formula (I) is preferably rinsed off with water after the application time.

According to a further preferred embodiment, the cosmetic formulation containing at least one compound according to formula (I) remains on the hair until the next hair cleansing.

### EXAMPLES

The following cosmetic formulations were prepared, in which at least one compound according to formula (a) was used as the active anti-dandruff ingredient. The quantities in the tables refer to wt %.

### 1) Shampoo

| | 1 | 2 | 3 |
|---|---|---|---|
| Anionic surfactant | 1-15 | | |
| Sodium laureth sulfate | | 1-15 | 3-11 |
| Compound of formula (I) | 0.05-5 | | |
| Methylheptyl glycerin | | 0.05-5 | 0.1-4 |
| Amphoteric surfactant | 0-10 | | |
| Cocamidopropylbetain | | 0-10 | 1-7 |
| Cationic polymer | 0-3 | | |
| Guar hydroxypropyl | | 0-3 | 0.01-1 |
| trimonium chloride | | | |
| Water and optionally | up to 100 | up to 100 | up to 100 |
| further auxiliaries | | | |
| and active ingredients | | | |

### 2) Hair Rinse

| | 1 | 2 | 3 |
|---|---|---|---|
| Ester oil | 0.6-1 | | |
| Isopropyl myristate | | 0.6-1 | 0.8-1 |
| Esterquat | 0.3-0.6 | | |
| Distearoylethyl | | 0.3-0.6 | 0.4-0.5 |
| hydroxyethylmonium | | | |
| methosulfate | | | |
| Emulsifier | 0.4-1 | | |
| Ceteareth-20 | | 0.4-1 | 0.5-0.8 |
| Fatty alcohol | 4-6 | | |
| Cetearyl alcohol | | 4-6 | 4-5 |
| Compound of formula (I) | 0.05-5 | | |
| Methylheptyl glycerin | | 0.05-5 | 0.1-4 |
| Cationic polymer | 0-3 | | |
| Guar hydroxypropyl | | 0-3 | 0.01-1 |
| trimonium chloride | | | |
| Water and optionally | up to 100 | up to 100 | up to 100 |
| further auxiliaries | | | |
| and active ingredients | | | |

### 3) Hair Tonic

| | 1 | 2 | 3 |
|---|---|---|---|
| Solvent | 15-50 | | |
| Ethanol | | 15-50 | |
| Isopropanol | | | 30-50 |
| Compound of formula (I) | 0.05-5 | | |
| Methylheptyl glycerin | | 0.05-5 | 0.05-5 |
| Water and optionally | up to 100 | up to 100 | up to 100 |
| further auxiliaries | | | |
| and active ingredients | | | |

The anti-dandruff efficacy and the skin-soothing efficacy of compounds according to formula (I) were investigated.

The efficacy against dandruff was investigated by is of a suspension test. Various formulations were incubated for a certain time (≤60 min) together with the yeast Malassezia furfur, which was present in cell counts of 105-106 CFU/mL, at 37° C. in a buffer solution.

After incubation, the cell count of the yeast was determined by is of a culture method (growth on Sabouraud Agar). The difference between this value and the respective initial value is a measure of the microbicidal efficacy of the respective anti-dandruff compound.

One of the causes of dandruff formation is the excessive growth of various microorganisms on the scalp. Yeasts of the genus Malassezia play a particularly important role here. The growth-inhibiting effect of various anti-dandruff compounds in this experiment therefore indicates anti-dandruff efficacy (FIG. 1).

A healthy scalp forms a protective barrier against external influences, prevents dehydration, and serves as the basis for optimal hair growth. The formation of dandruff is significantly promoted by the colonization by fungi, whose metabolic products negatively influence the barrier of the scalp and lead to inflammatory reactions.

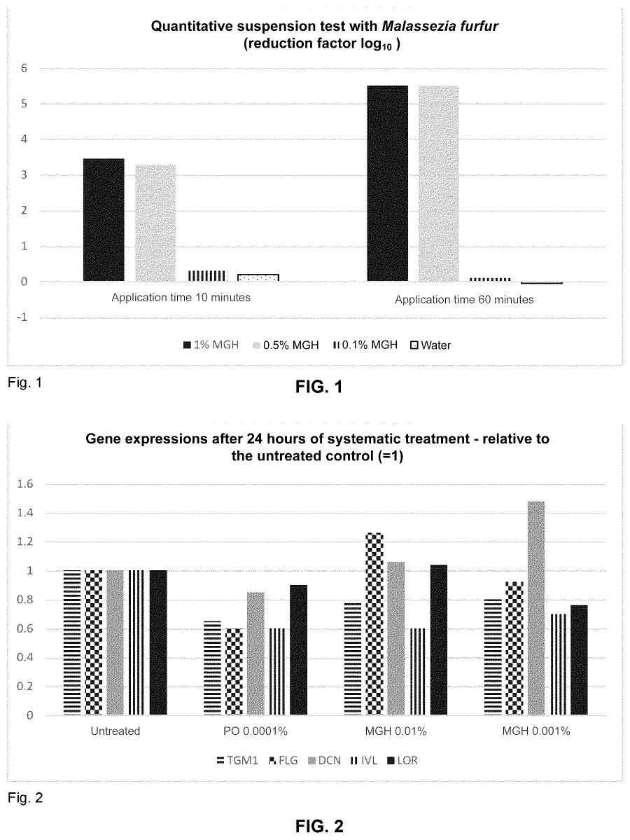

Using in vitro tests on artificial skin models, it was possible to show that common anti-dandruff agents such as Octopirox (Piroctone Olamine) negatively alter the barrier function. The gene expression of all investigated barrier markers (transglutaminase1, filaggrin, decorin, involucrin, and loricrin) was reduced by the treatment. Treatment with methylheptyl glycerin, on the other hand, did not lead to a significant decrease in gene expression, even at higher concentrations, and certain genes were even stimulated (FIG. 2).

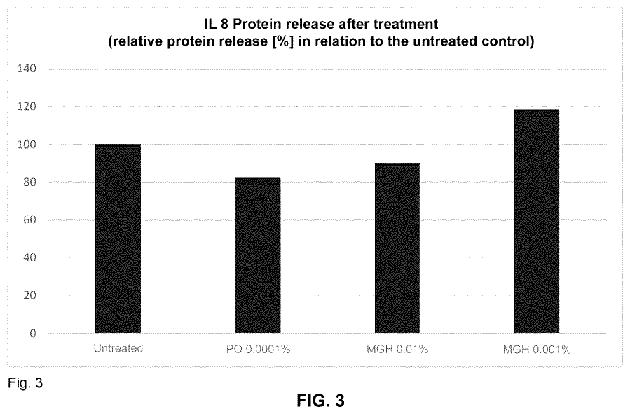

Furthermore, it was ascertained that methylheptyl glycerin (just like Octopirox) does not negatively influence the release of inflammatory markers. The release of interleukin 8 is within the untreated control range (FIG. 3).

In summary, it can be said that methylheptyl glycerin can be considered a milder alternative to the anti-dandruff agent, Octopirox. In addition to the killing effect on the Malassezia fungus, it provides additional advantages for maintaining the body's own barrier. MHG is particularly recommended for consumers with sensitive scalps (where the barrier is impaired and sebum production is elevated), as it can help to normalize the scalp.

The use of methylheptyl glycerin not only combats dandruff-causing fungi, but also has a balancing and soothing effect on the scalp.

What is claimed is:

1. A method for treating hair or scalp, comprising:
applying to the hair or scalp H₃C—(CH₂)₅—CH(CH₃)—O—CH₂—CH(OH)—CH₂OH (methylheptyl glycerin).

2. The method of claim 1, wherein the H₃C—(CH₂)₅—CH(CH₃)—O—CH₂—CH(OH)—CH₂OH (methylheptyl glycerin) is present in a cosmetic formulation.

3. The method of claim 2, wherein the cosmetic formulation is a shampoo.

4. The method of claim 2, wherein the at least one compound of formula I is present in the cosmetic formulation in an amount of 0.05-5 wt % relative to the total weight of the cosmetic formulation.

5. The method of claim 2, wherien the cosmetic formulation further comprises at least one surfactant in an amount of 0.05-20 wt % relative to the total weight of the cosmetic formulation.

6. The method of claim 1, wherein the hair or scalp is treated for a dandriff condition.

7. The method of claim 1, wherien the hair or scalp is treated to provide skin-soothing.

8. The method of claim 1, wherein the hair or scalp is treated to reduce or prevent the bacterial colonization of hair-covered skin surfaces with yeast fungi of the genus Malassezia.

9. The method of claim 1, wherein the hair or scalp is treated to reduce or prevent bacterial colonization of hair-covered skin with yeast fungi of the genus Malassezia furfur and/or Malassezia globosa.
